(19) United States
(12) Patent Application Publication
(10) Pub. No.: US 2021/0393818 A1
(43) Pub. Date: Dec. 23, 2021

(54) Embedded ultraviolet disinfection of aircraft interiors

(71) Applicant: Andrew S. Kanigowski, La Crescenta, CA (US)

(72) Inventor: Andrew S. Kanigowski, La Crescenta, CA (US)

(21) Appl. No.: 16/873,737

(22) Filed: Jun. 22, 2020

(51) Int. Cl. A61L 2/10; B64D 11/06; B64D 11/00; A61L 2/26

(52) U.S. Cl. CPC A61L 2/10; B64D 11/06; B64D 11/003; A61L 2202/25; A61L 2/26; A61L 2202/11; B64D 11/00151

(57) ABSTRACT

Presentation of an improved method of applying an ultraviolet light for disinfecting of aircraft interiors between flights. This novel system involves applying said UV light from the UV LEDs permanently embedded in the passenger seats, and/or in the IFE monitors, and/or in the overhead bins and/or in the ceiling, all connected to a remote on-off switch, to provide electric power to disinfect the entire cabin at once.

This invention allows for a significantly increased amount of time as well as target optimization of the UV light focused onto cabin surfaces compared to a push-card system with UV lights, which slowly and unevenly disinfects the aircraft interior, only one row of seats at a time.

The subject invention results in more effective disinfection, safer interior for the next load of passengers, cost-savings by eliminating push-cart operators, and shortening aircraft on ground down time, due to total cabin being disinfected at once, increasing the number of possible daily flight cycles, resulting in increased overall revenue for aircraft operators.

### REFERENCE TO RELATED APPLICATIONS

This is an international patent application under the Patent Cooperation Treaty which is based upon my parallel disclosure, including: the functional description and drawings submitted to the US Patent & Trademark Office, on the same filing date.

### TECHNICAL FIELD

This patent application covers a new and original design in the statutory class of machines. This invention relates to the mechanism of applying UV light for disinfecting of aircraft interiors before the next flight with a new load of passengers. More specifically, this invention involves a unique method of applying ultraviolet light to the aircraft seat surfaces, which were in direct body contact from human seat occupants on the prior flight(s).

### BACKGROUND ART

There are various methods of cleaning aircraft seats body-contact areas with various disinfectants. One of the closest methods to this invention is when the cleaning crew/person is walking the aisle, pushing a cart with above-the-seat positioned arms, emitting the UV light on the top of seats from above. But there are at least three major shortcomings with this method:

1. it requires an operator, a cleaning person(s) walking the aisles, often in hundreds of aircraft, several times every day, between each and every flight (very expensive labor cost).

2. it limits the time when the UV light is illuminating over each seat separately, resulting in very limited percentage of killed bacteria and viruses (means many viruses are still surviving).

3. it increases the downtime for the aircraft due to the manual labor, potentially decreasing the number of cycles for the aircraft per day resulting in lost revenue for the operator.

### DISCLOSURE OF INVENTION

This design innovation is related and directed to the permanently installed (embedded) UV lights to be located and installed at:
either on the aircraft seat upper structural components (eg. on the seat backrest structure).
or in the IFE (in-flight entertainment) video monitors,
or in the PSU (passenger service units) installed in the overhead bins or ceiling (in a similar fashion to the existing reading lights in the overhead bins).

When these permanently installed (embedded) UV lights are installed in the aircraft, then the disinfection starts with a flip of a remote on-off switch, by the flight attendant, when the airplane is empty (no passengers) between the flights. The switch could be safety proofed by being tied to the aircraft parking brake, allowing activation (disinfection) only upon the parking brake being set.

Such improved Embedded UV Disinfection System eliminates the need for expensive employment and training of numerous cleaning operators, to manually push the food-cart type devices in the aisles, while significantly increasing the time the UV light is illuminating on said body-contact areas of the seats, resulting in significantly longer “killing time” of any viruses laying on the seat.

This invention will result in much better (more effective) disinfection, and in a much safer flight for the next load of passengers.

As shown on the submitted drawings, the UV light can be emitted from any one, or from any combination of the two sources, or even from all of the following sources, for maximum protection:
from the upper area of the seat backrest (with said UV light directed downward and aftward.
from the IFE monitor (with UV lights embedded in the monitor, in the controlled direction)
from the overhead PSU (passenger service unit), or from the aircraft cabin ceiling area, and with UV light directed mainly downward, toward the seat exposed surfaces.

Again, this UV light will be directed toward the seat exposed surfaces, to sterilize them, when the seats are not occupied between the flights.

It is the objective of this invention to provide a simple, lightweight, very dependable, and inexpensive method for maximum disinfection of aircraft seats, with said embedded UV light system that is ‘user-friendly’, easy to operate, more effective, and safe for use before each flight, for a much better protection of the flying public (otherwise exposed to international viruses and quick spread of infections (like the spread of the COVID-19 worldwide).

### THE BEST MODE FOR CARRYING OUT THE INVENTION

The attached drawings are self-explanatory. The UV lights can be installed in any single one, or in any combination of the following locations:
in the upper area of the seat backrest,
in the IFE monitors,
in the overhead bins mounted PSUs.

### BRIEF DESCRIPTION OF DRAWINGS

In the drawings, all of the above three (3) basic suggested locations are shown but it is not to be construed as the only locations. For example, the UV lights can be installed in the ceiling of the aircraft cabin as well, for as long as the UV light is directed down toward the top-exposed seat surfaces.

FIG. 1 shows side and top view of seats, with UV light(s) mounted in the upper area of the seat backrests, and illuminating the UV light backward and down, to the body contact areas of the seats positioned immediately behind (to the next row seat).

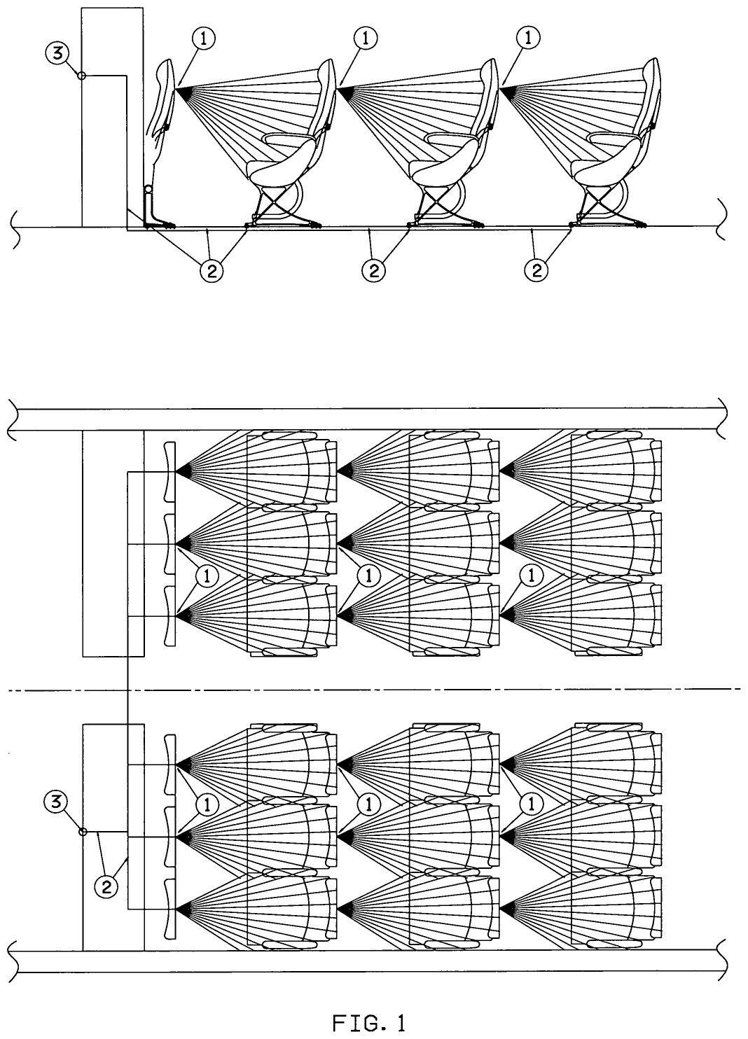

FIG. 2 shows side and top view of seats, with UV light(s) mounted inside or near the IFE video monitors (and directed similar to FIG. 1 above)

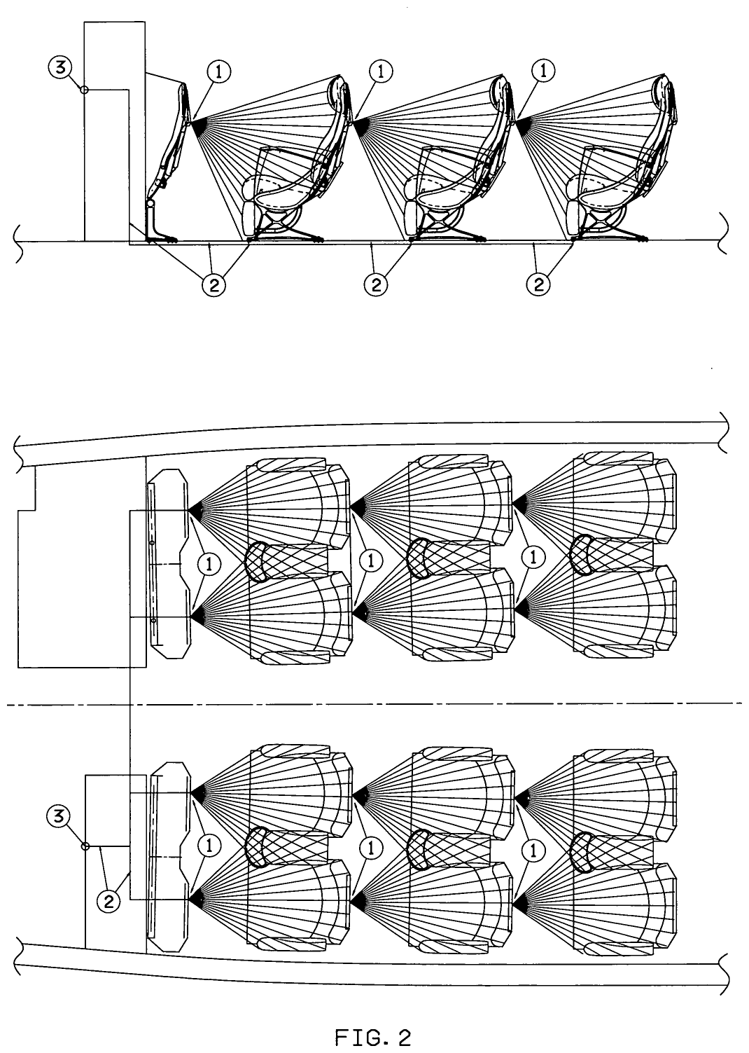

FIG. 3 shows side and top view of the cabin, with the UV lights installed in the overhead PSU units, or in the overhead bins, or in the overhead ceiling area to illuminate the seats from above

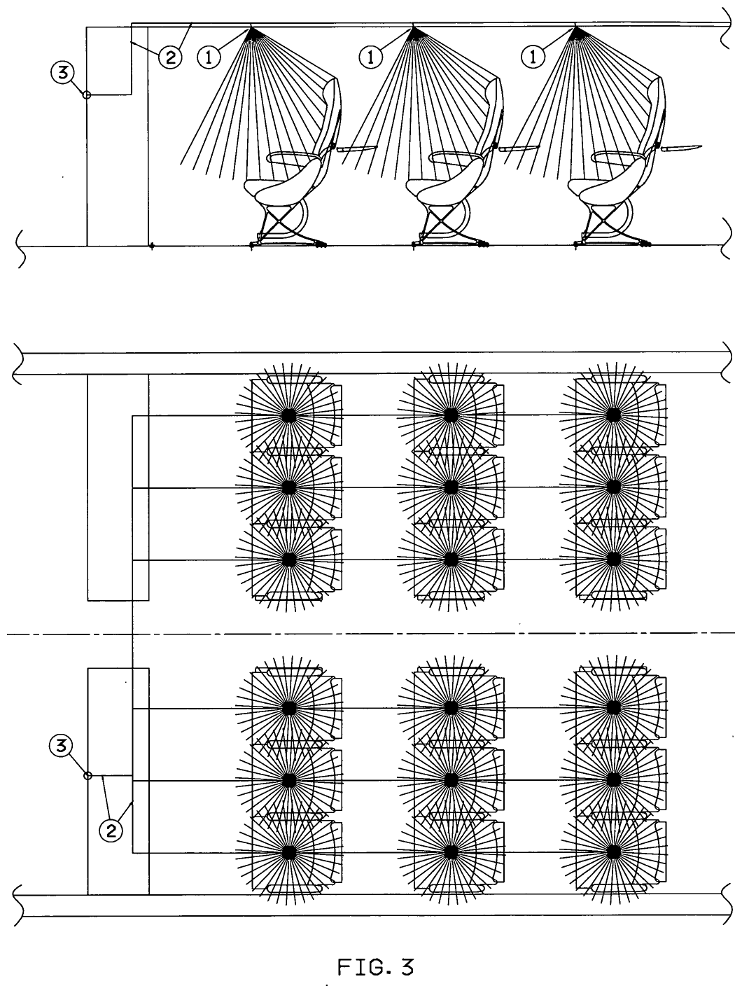

### NUMERALS IN DRAWINGS

Balloon 1: UV light bulb (or source) and approx. direction of the UV rays
Balloon 2: Electrical wiring (in the floor or in the ceiling, or in the side walls)
Balloon 3: Remote On-Off Switch in the Control Panel area (from the electric power source).

### INDUSTRIAL APPLICABILITY

The new Aircraft Cabin Embedded UV Disinfection System is to be designed, built, flight-tested and certified by FutureFlite, Inc., having FAA-PAH (Production Approval Holder) Privileges for a long time, and having 40-years hands on experience in the aircraft interiors business world.

Although the description above and drawings submitted contain many specificities, these should not be construed as limiting the scope of this invention but as merely providing illustrations to some of the presently preferred embodiments of this innovation. For example, the UV light can be directed both slightly forward, not only backward, and with the remote on-off-button not only near the front-end of the cabin, but even from the outside of that cabin of aircraft.

In yet another embodiment, it may be provided for disinfection of the passenger seats in bus, train, or in sea-going ferries, or in ocean-going cruise ship cabins.

Thus, the scope of this invention should be determined by the appended claims and their legal equivalents, rather than by the examples given.

I claim:

1. An improved aircraft cabin disinfection system adapted for disinfection of aircraft seats between flights, and comprising:
a) a passenger seat having a backrest,
b) a UV light installed in upper area of said backrest,
c) an electric power source wired to said UV light
d) a remote on-off-switch to operate said UV light whereby such improvement provides a quick and easy disinfection of aircraft seats when they are not occupied between flights.

2. An improved aircraft cabin disinfection system adapted for disinfection of aircraft seats between flights, and comprising:
a) a passenger seat having a backrest,
b) an IFE In-Flight Entertainment video monitor installed in said backrest,
c) a UV light installed in said IFE monitor,
d) an electric power source wired to said UV light,
e) a remote on-off-switch to operate said UV light whereby such improvement provides a quick and easy disinfection of aircraft seats when they are not occupied between flights.

3. An improved vehicle cabin disinfection system adapted for disinfection of passenger occupied areas in bus, train, ferry, or cruise ship, before these vehicles are boarded for the next trip, and comprising:
a) an occupant seats or beds with passenger body-contact areas,
b) an overhead storage bin or ceiling,
d) an embedded UV light installed in said overhead bun or ceiling,
e) an on-off-electric switch to operate said UV light when the cabin is not occupied
whereby such improvement provides a quick and easy disinfection of vehicle interior when it is not occupied between the next bus, train, ferry or cruise ship journey.
